(19) United States
(12) Patent Application Publication
(10) Pub. No.: US 2020/0404948 A1
(43) Pub. Date: Dec. 31, 2020

(54) METHOD OF SEQUENTIAL FUNGAL FERMENTATION OF LIGNEOUS RESOURCES

(71) Applicants: COMMISSARIAT A L'ENERGIE ATOMIQUE ET AUX ENERGIES ALTERNATIVES, PARIS (FR); HIPOFI, CLAMART (FR)

(72) Inventors: Agnès SAINT-POL, Clamart (FR); Jean-Philippe DESLYS, La Celle-Saint-Cloud (FR); Gwenaël POSTEC, Clamart (FR)

(73) Assignees: COMMISSARIAT A L'ENERGIE ATOMIQUE ET AUX ENERGIES ALTERNATIVES, Paris (FR); HIPOFI, Clamart (FR)

(21) Appl. No.: 16/979,473

(22) PCT Filed: Mar. 13, 2019
(86) PCT No.: PCT/EP2019/056234

§ 371 Date: Sep. 9, 2020

(30) Foreign Application Priority Data
Mar. 13, 2018 (FR) 1852151

(51) Int. Cl. A23K 10/12; C12N 9/24; C12N 9/26; C12N 9/62; C12P 17/12; A23K 10/32; A23K 20/142; A23K 20/189; A23K 20/174; A23K 50/75

(52) U.S. Cl. CPC A23K 10/12; C12N 9/248; C12N 9/2411; C12N 9/62; A23K 50/75; A23K 10/32; A23K 20/142; A23K 20/189; A23K 20/174; C12P 17/12

(57) ABSTRACT

The present disclosure relates to a method for transforming wood residues into an edible food product for a mammal, consisting of a sequence of fungal fermentations which make it possible to render ligneous resources edible; the invention also relates to the food product obtained by this method, and to the use thereof.

The present invention relates to the enhancement of coproducts of the forestry industry; more specifically, it relates to the development of a method improving the digestibility of wood in order to render it suitable for consumption by mammals. The present invention therefore also relates to a food having advantageous nutritional properties, in particular for farm animals.

Due to its significant lignin content which render it indigestible (20 to 30%), wood almost has no food usage. The present invention provides an original method consisting of a sequence of fungal fermentations rendering ligneous resources edible. In practice, a first fermentation at ambient temperature with a wood destroying edible fungus for a few weeks makes it possible to obtain a substrate rich in fungal compounds of interest, and lignin-depleted. Stopping this fermentation at the optimum moment by a suitable method makes it possible to obtain a substrate capable of a second fermentation of a few days by an edible fungus with a high added value. The product thus obtained contains fungal enzymes and compounds of interest and can be used directly as a food supplement.

France has the fourth largest forest in Europe in surface area (17 million hectares in mainland France), behind Sweden, Finland and Spain, but harvest, since the 1980s, has not exceeded half of the annual production of wood (Alexandre, 2017). Therefore, there are between 30% and 40% of the French and European surface areas which are covered with forests. In France, 70% of forests consist of deciduous trees and 30%, evergreen trees with a preponderance of oak trees over all of the species represented and an annual gross increase of French forest by 85 million m³ in wood with strong trunks between 2001 and 2009 (Agreste 2012); this figure even comes to 120 million m³ if branch wood is included. About 55 million m³ have been harvested each year during the same period for use distributed between energy wood and timber and industrial wood (Agreste 2012).

The first transformation methods associated with this operation generate residues or coproducts in a massive quantity. The industrial yield in a sawmill which manufactures boards is about 50% (Alexandre, 2017), the other half termed wood-related (barks, sawdust, chips, splinters, etc.) only finds low-added-value uses for which it would be useful to find new enhancement methods (Alexandre, 2017).

Lignin is the second most abundant renewable biopolymer on the Earth, and the only aromatic carbon source generated in nature (on average, 20% in hardwood and 30% in softwood) (Howard, Abotsi, L, & Howard, 2003). Its main functions are to provide rigidity, impermeability to water and great resistance to decomposition. Lignin is a three-dimensional amorphous polymer composed of methoxylated phenylpropane structures and isolated lignins generally have a molecular weight of about 2000 to 5000 Da (Wertz, 2010).

Like biopolymer, lignin is unusual due to its heterogeneity and its lack of defined primary structure, it constitutes the “glue” which holds the cell walls together. Lignin polymers render the cell wall rigid and impermeable, allowing the transport of water and nutritional elements through the vascular system and protect plants from microbial invasion. Lignin is extremely resistant to degradation and, by forming bonds both with cellulose and hemicelluloses, it creates a barrier to all solutions or enzymes (Wertz, 2010), thus forming one of the major obstacles to the conversion of lignocellulose biomass into biobased fuels and chemical products.

In nature, the effective degradation of lignin during the phenomenon of wood rot is possible mainly through wood white rot basidiomycete fungi (Plácido & Capareda, 2015), which produce specific enzymes such as laccases, manganese peroxidase and lignin peroxidase. Numerous white rot fungi simultaneously attack lignin, hemicelluloses and cellulose, while other white rot fungi attack lignin selectively. Contrary to white rot fungi, brown rot fungi can degrade the polysaccharides of the wood, but not oxidised lignin. Ascomycetes are above all capable of degrading cellulose and hemicelluloses, but their capacity to degrade lignin is limited (Wertz, 2010).

Lignin has several applications of relatively low added value, such as:
fuel, supplying more energy when burned than cellulose;
additive in cement, in particular as a cement setting retarding agent;
additive in asphalt, in particular for its antioxidant features;
binder in food for animals to plasticise and hold the granules together;
additive in combustible granules based on the biomass (Wertz, 2010).

The developments of methods for treating and fermenting lignocellulose compounds have risen for several years, in particular for the production of biofuels, enzymes, pigments and secondary metabolites (Guerriero et al., 2016) (Dashtban, Schraft & Qin, 2009) (Soccol et al., 2017). The large majority of these developments are carried out from lignocellulose compounds from farming, such as straws, brans, cattle cakes (Soccol et al., 2017) and hardly any are based on the use of substrate from forest exploitation (Thomas, Larroche & Pandey, 2013) (Ferreira, Mahboubi, Lennartsson & Taherzadeh, 2016).

However, on a planet with finite resources and a continually growing population, the implementation of a circular economy with an optimisation of using bioresources for food purposes must be a priority (FAO, 2016). Farming land is not indefinitely extendable at the expense of forest, as otherwise, the lungs of the planet which would suffer.

The present invention therefore provides the development of a method which would allow, for the first time, to use wood for food purposes; this method consists of a sequence of fungal fermentations making it possible to render ligneous resources edible.

The present invention also relates to the product obtained by this method which has remarkable nutritional properties through its composition of vitamins, minerals, essential amino acids and particularly, through its content of high-value enzymes for animal food, in particular xylanases, amylases and proteases.

In practice, a first fermentation at ambient temperature with a wood destroying edible fungus for a few weeks makes it possible to obtain a substrate in rich in fungal compounds of interest and lignin-depleted (similar to the lignin level found in straw). Stopping this fermentation makes it possible to obtain a substrate capable of a second fermentation of a few days by an edible fungus with high added-value. This first fermentation allows the development of an ascomycete such as Aspergillus oryzae, in the context of a second fermentation, on sawdust fermented under conditions where the addition of nitrogenated nutritional elements is zero. From the second fermentation, a stabilisation of secreted enzymes contained in the fermented product obtained is carried out by low-temperature dehydration.

Thus, the present invention relates to a method for transforming wood residues into a food product which is edible for a mammal comprising steps of:

1) optionally, pre-treatment of wood residues such as a grinding and/or reduction of the tannin content of the wood and/or the addition of an alkalinising mineral supplement and/or a heat treatment intended to remove possible contaminants and/or a gentle heat treatment followed by a lactic fermentation;

2) first fermentation of a substrate composed of wood residues and optionally comprising from 1 to 5% by dry weight of an alkalinising mineral supplement, by a wood destroying edible fungus for a suitable duration corresponding to the maximum colonisation of the substrate before fructification by said fungus; this duration varies according to the fungus layer used and the temperature implemented; as an example, according to an optimised embodiment for Pleurotus osteratus, this first fermentation is conducted for 30 to 40 days at a temperature of 28° C.; if a lower temperature is implemented, the fermentation time will need to be extended;

3) stopping the first fermentation by heat inactivation of said wood destroying edible fungus and grinding of the product obtained from said first fermentation; preferably, the grinding is carried out before the heat inactivation;

4) second fermentation of the product obtained in step 3) by a fungus of the Aspergillus genus for a suitable duration corresponding to the maximum colonisation of the product obtained in step 3) before sporulation of said fungus of the Aspergillus genus; as an example, according to an optimised embodiment for Aspergillus oryzae, this second fermentation is conducted for 3 to 4 days at an optimal growth temperature at 30° C.; if a lower temperature is implemented, the fermentation time will need to be extended;

5) optionally, stabilisation of the product obtained from said second fermentation by dehydration.

The substrate, or starting material, of the method according to the invention comprises wood residues such as shavings (residue size between 1 mm and 2 cm), sawdust (residue size between 1 mm and 2 cm), or also wood meal (residue size between 20 μm and 1 mm); according to the quality of the wood and its digestibility by the wood destroying fungus, the substrate can also comprise larger wood pieces (of a size greater than 2 cm).

In order to implement the first fermentation of the method according to the invention, it is, however, preferable to have wood residues of which the maximum size is less than or equal to 2 cm; thus, if the wood residues available have a size greater than 2 cm, prior grinding is carried out. Any grinding technique making it possible to reduce the size of the wood residues can be used.

According to a particular embodiment of the invention, it is advantageous to mix wood residues having different sizes, for example, between 40 and 80% by weight of sawdust and between 20 and 60% by weight of wood meal possibly in the presence of larger pieces; this difference in grain size favours a satisfactory aeration of the substrate without it being necessary to proceed with a mechanical stirring during the first fermentation.

Any wood species can be used for the implementation of the method according to the invention whether softwood or hardwood trees as demonstrated in the following experimental section; preferably, these are species utilised industrially. As an example, species of softwood trees which can be used, are firs, spruces, maritime pines, Douglas firs; those of hardwood trees which can be used, are oaks, poplars, beeches, acacias, chestnuts, nannyberries.

According to the species of the tree, from which the wood used comes, it can be preferable to reduce the content of tannins of its wood; tannins indeed contribute to the defence system that plants have developed against fungi and limit the digestibility and the absorption of proteins from food rations of farm animals (Gilani et al., 2017), (Sharma & Arora, 2015). For this, wood residues are mixed with water and heated to a temperature between 50 and 120° C., preferably to about 90° C. water is then removed by filtration, for example, on cellulose filter until obtaining a moisture level between 55 and 70% (Girmay et al., 2016), (Hoa & Wang n.d.).

The aim of the first fermentation is mainly the degradation of the lignin present in wood residues and the release of nutrients which will be consumed in the context of the second fermentation.

According to a particular embodiment, the substrate of the first fermentation of the method according to the invention is prepared by adding, to the wood residues, an alkalinising mineral supplement in a quantity between 1 and 5% by dry weight, preferably between 2 and 3% by dry weight, also preferably about 2.5% by dry weight with respect to the total weight of wood residues used in the substrate.

Adding the alkalinising mineral supplement has proved to be advantageous in pre-treatment of woods which are difficult to digest, for example, those used for their rot-proof character (see table 1 in the experimental section). These woods are generally those of hardwood, for example, oaks and acacias. When it is added to wood residues, the alkalinising mineral supplement is preferably introduced before a possible wet heating such as described earlier to favour the deconstruction of the substrate.

The mineral supplement is alkalinising, i.e. that it has a basic pH, more specifically a pH greater than or equal to 8, preferably, greater than or equal to 9, also preferably, greater than or equal to 10, before mixing with wood residues and that it allows the preparation of a substrate, of which the pH is at least 7, preferably 8 before heating. According to this particular embodiment, the alkalinising mineral supplement comprises at least one alkalinising mineral which can be, in particular, selected from potash, calcium carbonate, lye, calcium hydroxide, sodium hydroxide or potassium hydroxide.

Preferably, the alkalinising mineral supplement consists of ashes from the combustion of coproducts of the wood industry (wood heating ashes) thus making it possible to optimise their recycling.

Preferably, the alkalinising mineral supplement represents an input of:
170 to 330 kg/t of calcium (expressed in the form of CaO),
20 to 60 kg/t of potassium (expressed in the form of K₂O),
25 to 46 kg/t of magnesium (expressed in the form of MgO),
10 to 61 kg/t of phosphorus (expressed in the form of P₂O₅),
metals, including Mn, Fe, Cu, Zn which are cofactors of digestive enzymes secreted by fungi, in variable proportions,
   and has a pH, before mixing with wood residues, between 10 and 13.

Preferably, the substrate comprising wood residues and possibly an alkalinising mineral supplement is treated to remove possible contaminating microorganisms, even to reinforce its alkalinising properties, if necessary; according to a particular embodiment, the substrate is heated before the implementation of the first fermentation; the heating means is selected by a person skilled in the art, in particular according to the substrate volume to be treated.

An alternative pre-treatment method consists of directly treating wood residues with a gentle heat treatment using the principle of tyndallisation with a sequence of 60 to 80° C. at the core for at least one hour, two to three consecutive times at 24-hour intervals, by letting naturally cool in the interval, which makes it possible to destroy the vegetative forms of the contaminants and to force the germination of the spores before destroying the new vegetative forms without the possibility of any new sporulation; this gentle heat treatment is followed by a lactic fermentation to limit the risks of subsequent uncontrolled contamination while remaining under “rustic” conditions (for example, with aspersion of a mixture of bacterial strains of type Streptococcus thermophilus and Lactobacillus delbrueckii subsp. Bulgaricus and/or any other strain of lactobacillus of food interest with a fermentation at about 43° C. for about 8 hours). A substrate of wood residues thus prepared is both cleared of its natural contaminants and better protected from undesirable contaminants during the implementation of the first fermentation by a wood destroying fungus strain.

According to a particular embodiment, no other pre-treatment than grinding, reducing the tannin content, adding an alkalinising mineral supplement, the heat treatment intended to remove the possible contaminants and/or a gentle heat treatment followed by a lactic fermentation is applied to the wood residues.

The substrate has a moisture content between 50 and 70%, preferably between 60 and 70%.

The substrate is inoculated by a wood destroying edible fungus in its primary mycelium form which can in particular be selected from Pleurotus ostreatus, Pleurotus pulmonarius, Hypsizygus ulmarius, or also Agaricus blasei and Agaricus braziliensis; preferably, this is Pleurotus ostreatus.

According to a particular embodiment, and in order to facilitate the starting of the first fermentation, the wood destroying edible fungus is pre-cultivated on a suitable culture medium before being seeded on the substrate.

The implementation conditions of this pre-culture are known to a person skilled in the art; the pre-culture can, for example, be carried out on wheat, brewer's grains, rice or also a mixture of rice, straw and/or wood with the addition of lime or calcium carbonate.

The substrate is inoculated with between 10 and 20% by dry weight, preferably of the order of 20% by dry weight of the preculture of the wood destroying edible fungus and is then maintained at an optimal growth temperature for the wood destroying edible fungus used; for example, the culture temperature is between 20 and 30° C., preferably of the order of 28° C. for the basidiomycetes Pleurotus ostreatus (Hoa et al., n.d.), Pleurotus pulmonarius (Belletini et al., 2017) and Hypsizygus ulmarius, or between 25 and 35° C., preferably 30° C. for Agaricus blazei or braziliensis (Colauto et al., 2008).

The duration of the fermentation is conducted until the complete colonisation of the substrate by the wood destroying edible fungus.

According to a particular embodiment, the culture medium of the first fermentation (substrate and population of wood destroying edible fungus) is ground and/or mixed at least once during the first fermentation to standardise the development of said fungus.

Surprisingly, the treatment of wood residues by the first fermentation according to the invention allows the growth and the development of a second fungus of the Aspergillus genus on a substrate on which it cannot normally be grown.

The stopping of this first fermentation preferably occurs before the fructification of the wood destroying edible fungus such that the first fermentation is only carried out with the primary mycelium of the wood destroying edible fungus.

From this first fermentation, all of the culture is reground/homogenised to serve as a base for the second fermentation.

Said wood destroying edible fungus is then inactivated by heat treatment. A person skilled in the art will know how to select the most suitable heat treatment; as an example, according to an embodiment suitable for an industrial implementation of the method of the invention, the heat treatment is conducted at about at least 70° C. in a wet medium (for example, in a counter-current device, by treatment with water vapour or also by treatment with intense heat) for about 1 hour.

After grinding and inactivation, the culture from the first fermentation (substrate of the second fermentation) is optionally enriched with a second mineral supplement to satisfy the nutritional needs of the fungus of the Aspergillus genus; this optional enrichment can be implemented when the first fermentation is not allowed a release of minerals in a sufficient quantity for the growth of the Aspergillus fungus.

For the implementation of this optional variant of the method, and practically, this second mineral supplement comprises at least one phosphate salt; it can also comprise a magnesium, sulphate and/or potassium source; it is added to the substrate of the second fermentation in a quantity between 1 and 5% by dry weight, preferably between 2 and 3% by dry weight, also preferably about 2.5% by dry weight with respect to the total weight of substrate of the second fermentation.

The substrate of the second fermentation is seeded with a quantity of spores of a GRAS (“Generally Recognised As Safe”) fungus species and commonly used in the preparation of food products, of the Aspergillus genus, between 5·10⁵ and 2·10⁶/g of substrate. The fungus species of the Aspergillus genus is in particular selected for its capacity to secrete enzymes of the hemicellulose type.

The water content of the substrate of the second seeded fermentation is then, if necessary, adjusted to a value between 55 and 75%, preferably between 60 and 70%, also preferably to about 65%.

Preferably, the species used for this second fermentation is selected from Aspergillus oryzae, Aspergillus niger, Aspergillus sojae or also Aspergillus awamori; the second fermentation can also be carried out with a mixture of at least two species of Aspergillus, for example A. oryzae and A. awamori; also preferably, this is Aspergillus oryzae.

According to a particular embodiment using the species Aspergillus oryzae, the Aspergillus oryzae spores have been collected beforehand after culture, for example on PDA medium containing 0.6M of KCl in order to stimulate the sporulation (Song et al., 2001).

As an example, when the second fermentation is implemented with Aspergillus oryzae, it is stopped after 2 to 3 days, preferably 3 days, of incubation at a temperature between 25 and 40° C., preferably between 28 and 30° C. The fermented product is thus recovered.

The aim of this second fermentation is to increase the overall fungal biomass and the production of enzymes of interest for animal food (in particular, xylanase, amylase, protease, phytase).

According to a particular embodiment, the second fermentation is stopped when the maximum secretion of xylanases, amylases and/or proteases is reached.

The product obtained from the second fermentation is stabilised by dehydration, this method advantageously allows a stabilisation of the enzymes of interest on the fermented product, which serves as an immobilisation support. For this, it can, for example, be placed after homogenisation by mechanical stirring in a chamber at about 24° C. until obtaining a water content less than or equal to 12%, preferably between 10 and 12% (corresponding to an activity of water (aw) less than 0.6 and preventing the growth of microorganisms), then stored in the cold or at ambient temperature. Any other drying method known to a person skilled in the art, like for example, lyophilisation, could also be implemented.

The fermented product obtained from the method according to the invention can be used directly and in its entirety as a food supplement for animal or human food, preferably for animal food.

The present invention also relates to a fermented food product which can be obtained by the method according to the invention and to its use, in particular as a food supplement for farm animals.

The food product according to the invention is characterised by a particularly useful nutritional composition, in particular for animal food.

Indeed, the enzymes secreted by filamentous fungi in the presence of lignocellulosic or starch-rich substrates are widely used in animal food to improve the digestibility of food and to increase the growth performances mainly of monogastric farm animals (Asmare, 2014).

In particular, xylanase, protease, amylase, glucanase and phytase activities are the most sought activities for animal food (Shallom & Shoham, 2003; Kuhad et al., 2011; Asmare, 2014).

The benefit of such an enzymatic cocktail has been evaluated beforehand on the growth of chickens (Cowieson and Ravindran, 2008). These have been fed for 21 days with a maize and soya-based food, complemented or not by an amylase, protease and xylanase cocktail (Avizyme® of the company Danisco). The added enzymatic activities have been 300 U of xylanase, 400 U of amylase, and 4000 U of protease per kg of food. The weight gain observed for the sample of chickens, of which the food rations have been complemented by this cocktail has been evaluated at 2% and the conversion of food (calculated by the consumption/weight gain ratio) has been increased by 8% compared with a sample of chickens not receiving this supplement; these data show the potential to improve these types of enzymatic cocktails on the digestibility of these supplements.

In the present case, the method according to the invention makes it possible to obtain a food product which advantageously comprises the following enzymes:
between 5 and 10 U of xylanases/g of dry fermented product,
between 5 and 10 U of amylases/g of dry fermented product, and
between 30 and 100 U of proteases/g of dry fermented product.

In addition to providing enzymes, the fermented product according to the invention is naturally rich in mycelium, the filamentary structure of filamentous fungi surrounded by a wall. These walls are complex structures, mainly composed of polysaccharides including the most abundant, beta-glucans (Bowman & Free, 2006), have recognised immuno-modulating properties (Volman, Ramakers & Plat, 2008).

Advantageously, the food product according to the invention also contains vitamins, in particular vitamin B3 in a content between 20 and 40 mg/g of food product.

This food product also has the advantage of containing essential amino acids at a level of, expressed in mg/g, of total proteins:
between 10 and 15 mg of histidine,
between 30 and 45 mg of isoleucine,
between 40 and 65 mg of leucine,
between 20 and 30 mg of lysine,
between 10 and 15 mg of methionine,
between 25 and 40 mg of phenylalanine,
between 12 and 19 mg of tyrosine,
between 35 and 54 mg of threonine,
between 25 and 40 mg of valine, and
between 8 and 12.5 mg of tryptophan,
   with a content of total proteins between 2.5 and 4.0% preferably, about 3.0% by weight with respect to the dry weight of said product.

Finally, thanks to its production method, this food product has a good digestibility as its lignin, cellulose and hemicellulose contents are reduced. The lignin content of the food product according to the invention, of course depends on the lignin content of the starting product (wood residues); the method according to the invention makes it possible to reduce the lignin content of the starting product of at least 30%, preferably of at least 40% and also preferably of at least 50%. As a comparison, the lignin content of the food product according to the invention is comparable to that of straw.

The present invention therefore relates to a food product comprising:
between 5 and 10 U of xylanases/g of dry food product;
between 5 and 10 U of amylases/g of dry food product;
between 30 and 100 U of proteases/g of dry food product;
between 20 and 40 mg of vitamin B3/g of dry food product;
an amino acid profile comprising between 10 and 15 mg of histidine, between 30 and 45 mg of isoleucine, between 40 and 65 mg of leucine, between 20 and 30 mg of lysine, between 10 and 15 mg of methionine, between 25 and 40 mg of phenylalanine, between 12 and 19 mg of tyrosine, between 35 and 54 mg of threonine, between 25 and 40 mg of valine and between 8 and 12.5 mg of tryptophan/g of total proteins of said food product;
a lignin content less than 18%, preferably less than 15%, also preferably less than 12.5% and preferably less than 11% by weight.

Preferably, this product comprises a total protein content between 2.5 and 4.0%, preferably of about 3.0% by weight with respect to the dry weight of said product.

The present invention also relates to the use of the food product as a food supplement by adding 3 to 4% by weight in an animal food ration.

Thus, the solid phase sequential fermentation method according to the present invention allows the use of ligneous resources as a substrate and the incorporation of the fermented product in toto in food and in particular, animal food. Regulation no. 68/2013 of 16 Jan. 2013 of the European Commission establishes the list of raw materials for animal food ((EU) REGULATION NO. 68/2013 OF THE COMMISSION of 16 Jan. 2013 relating to the catalogue of raw materials for animal foods, http://eur-lex.europa.eu/eli/reg/2013/68/oj) and including here wood lignocellulose, obtained by mechanical transformation of natural raw timber (section C, table and line 7.8.1), hardwood or wood fibre (section C, table and line 7.14.1) and vegetal wood charcoal (section C, table and line 7.13.1). However, the lignocellulosic nature of wood, its rigidity and its lignin content do not make wood a candidate frequently selected for animal food, as well as for the fermentation of lignocellulose compounds. The method according to the invention corrects this drawback.

Another major advantage of the sequential fermentation method according to the invention is based on the use of the fermented product as a support for immobilising/adsorbing secreted enzymes. Indeed, usually, the incorporation of the fermented product in animal food requires its microbiological and enzymatic stabilisation for the conservation of the product. The immobilisation of the enzymes on an insoluble support of organic and inorganic origin is known and pure substrates of glucidic nature, such as cellulose, starch, agar-agar, alginates have been used (Krajewska, 2014). The method developed here provides the use of the fermentation product composed of dehydrated residual lignocellulose and mycelium as an immobilisation support being substituted for the traditional supports described above. Surprisingly, the enzymatic activity measured is very well conserved and stable at ambient temperature after simple dehydration of the fermented product.

### FIGURES

FIG. 1: A. First fermentation: growth of Pleurotus ostreatus on oak sawdust after 40 days of incubation at 28° C. in the absence of or in the presence of mineral supplement (CM). B. Second fermentation: growth of Aspergillus oryzae after 3 days of incubation at 30° C. after different first fermentation conditions.

FIG. 2: A. Growth of Aspergillus oryzae on oak sawdust not fermented by Pleurotus ostreatus with and without combination with a mineral supplement and/or a nitrogenated supplement (in the form of protein, in the present case) (the insert has the growth of A. oryzae after sequential fermentation). B. Growth of Aspergillus oryzae on a culture medium containing 1.5% of glucose, 0.6% of NaNO₃, 0.15% KH₂PO₄, 0.05% MgSO₄, 0.05% KCl and minerals in trace form (Mn, Co, Zn and Fe) adjusted to different pHs. C. Growth of Aspergillus oryzae on a minimum culture medium (1.5% of glucose, 0.6% of NaNO₃), complemented such as indicated in the figure and adjusted to pH 6.1.

FIG. 3: A. Growth of Pleurotus ostreatus on oak sawdust after 40 days of incubation at 28° C. after combination with different alkaline and/or mineral supplements. B. Development of Aspergillus oryzae following this first fermentation after 3 days of incubation at 30° C.

FIG. 4: Comparison of xylanase (A), amylase (B) and protease (C) activities secreted by Pleurotus ostreatus (from the first fermentation) (PO, clear grey) and by Aspergillus oryzae (from the second fermentation) (PO/AO, black), according to the percentage of mineral supplement added before the first fermentation. The activities are expressed in units (μmol of product generated/min)/g of dry fermented product.

FIG. 5: Comparison of xylanase (A), amylase (B) and protease (C) activities secreted by Pleurotus ostreatus (PO, clear grey) and by Aspergillus oryzae (PO/AO, black), after different culture times of Pleurotus ostreatus on oak sawdust combined with 2.5% of ash. The activities are expressed in units (μmol of product generated/min)/g of dry fermented product.

FIG. 6: Effect of the dehydration (A) and of the conservation (B and C) of the fermented product on xylanase, amylase and protease activities. (MC: moisture content).

FIG. 7: Enzymatic activities (A—amylase and xylanase and B—protease) measured from the sequential fermentation method using Aspergillus oryzae or Aspergillus awamori during the second fermentation.

FIG. 8: Enzymatic activities (A—amylase and xylanase and B—protease) measured from the sequential fermentation method using Aspergillus oryzae or Aspergillus awamori during the second fermentation, individually or in coculture.

FIG. 9: measurement of the laccase activity of the product from the first fermentation (histogram on the left) and that of the product from the second fermentation (histogram on the right).

### EXAMPLES

### Example 1—Sequential Fermentation Method of Oak Wood with Pleurotus ostreatus then Aspergillus oryzae According to the Invention

### 1. EQUIPMENT AND METHODS

1.1. Sequential Fermentation Method

Pre-Treatment of the Wood

The oakwood residues obtained in the form of sawdust have been coarsely ground using a blade grinder to obtain a minor fraction in the form of meal (about 20%). The aim is to reduce the size (between 50 μm and 1 mm) and the crystallinity of a fraction of the lignocellulose of the wood with the aim of increasing its exchange surface and thus to facilitate enzymatic degradation (Saritha et al., 2012; Ravindran & Jaiswal, 2015).

They have been then subjected to a pre-treatment by heating to 90° C. in an aqueous medium in order to extract a portion of the extractables including water-soluble tannins.

After filtration on cellulose filter until obtaining a moisture content between 55 and 70% (Girmay et al., 2016) (Hoa & Wang, n.d.), an alkaline mineral supplement (ash) can be added then the substrate is autoclaved.

First Fermentation

The substrate is thus inoculated by Pleurotus ostreatus on rice. The inoculated substrate is then kept at 28° C., optimum growth temperature (Hoa et al., n.d.) for a period between 30 and 40 days until complete colonisation of the substrate by Pleurotus ostreatus (Hoa & Wang, n.d.).

Second Fermentation

From this first fermentation, the culture is ground using a blade grinder with the aim of making it homogenous and to serve as a base for the second fermentation. Oyster mushrooms are then inactivated by heating (between 70° C. and 120° C.), then 2·10⁶ spores of Aspergillus oryzae are added per gram of dry fermented product with adjustment of moisture between 60 and 70%.

The Aspergillus oryzae spores have been collected beforehand after culture on PDA medium containing 0.6M of KCl in order to stimulate the sporulation (Song et al., 2001).

The culture is stopped after 3 days of incubation at 30° C., the fermented product is recovered.

Stabilisation

The fermented product is stabilised by dehydration: it is placed after homogenisation by mechanical stirring in a chamber at 24° C. until obtaining a moisture content of 11-12%, corresponding to an activity of water (aw) less than 0.6 and preventing the growth of microorganisms (Assamoi et al., 2009), then it is stored at 4° C. or ambient temperature.

1.2. Characterisation of the Fermented Food Product Obtained

1.2.1. Measurement of the Enzymatic Activities

Preparation of the Enzymatic Raw Extract

The enzymes secreted by fungi are isolated from the fermented product directly after the stopping the incubation period or after stabilisation. The equivalent of 0.1 g of dry fermented product is removed to an Eppendorf tube then placed in 2 ml of acetate buffer 50 mM pH 5.0 and stirred (incubator stirrer 150 rpm) for 30 minutes at 30° C. (Chancharoonpong et al., 2012). The supernatant containing the secreted enzymes is collected after centrifugation for 10 minutes at 10,000 g (4° C.).

Measurement of Xylanase, Amylase and Protease Activities

The xylanase activities are measured by using as a substrate, beech xylan at 1% in an acetate buffer 50 mM pH 5.0. Typically, 50 μl of raw enzymatic extract are added to 150 μl of substrate then incubated for 50 minutes at 50° C. (van den Brink et al., 2013). The appearance of reducing ends after enzymatic cutting is measured by colorimetric dosing at 405 nm after reaction with p-4-hydroxybenzhydrazide (Szilagyi et al., 2010).

The amylase activities are measured by using as a substrate, starch at 0.2% in an acetate buffer 50 mM pH 5.0. Typically, 50 μl of raw enzymatic extract are added to 150 μl of substrate then incubated for 50 minutes at 50° C. (van den Brink et al., 2013).

The protease activities are measured by using azocasein as a substrate as described (Janser et al., 2014) with a few modifications: 200 μl of enzymatic extract are added to 200 μl of azocasein at 0.5% in an acetate buffer 50 mM pH 5.0. The incubation is carried out for 1 hour at 55° C., then proteins are precipitated by adding 400 μl of 10% trichloroacetic acid. After 10 minutes in ice, the tubes are centrifugated at 10,000 g for 10 minutes. 100 μl of supernatant containing azopeptides and azo amino acids are transferred into a microplate containing 100 μl of NaOH 5M. The absorbance is measured at 428 nm to determine the protease activity of the raw extract.

Measurement of the Laccase Activity

The laccase activity is measured by using ABTS 0.2 mM (2,2′-Azino-bis(3-ethylbenzothiazoline-6-sulfonic acid)) as a substrate (Valiskovi& Baldrian, 2006) in an acetate buffer 50 mM pH 5.0. Typically, 20 μl of raw enzymatic extract are added to 140 μl of acetate buffer pH 5.0 and 40 μl of ABTS 1 mM. The enzymatic activity is then evaluated immediately by measuring absorbance at 420 nm, corresponding to the oxidation of ABTS by the laccase activity and, carrying out kinetics over 90 minutes.

1.2.2. Determination of Lignin and Beta-Glucan Contents

The determination of the lignin content has been achieved by gravimetry after acid hydrolysis: the sample undergoes a succession of attacks by different solutions (neutral detergent, then acid detergent) in a Fibertec-type device (Boiling for 1 hour). At the end of each attack, the sample is carefully rinsed, dried and weighed. An attack with a highly concentrated acid is thus carried out, and the sample containing the lignin fraction is dried then weighed to determine the lignin content compared with the dry starting weight.

The determination of the beta-glucan content is achieved after specific enzymatic hydrolysis. The samples undergo successive enzymatic digestions. The glucose contained in beta-glucans 1.3-1.6, is thus released and determined by ion chromatography.

### 2. RESULTS

The Development of Aspergillus oryzae on Wood Residues is Dependent on the Prior Development of Pleurotus ostreatus.

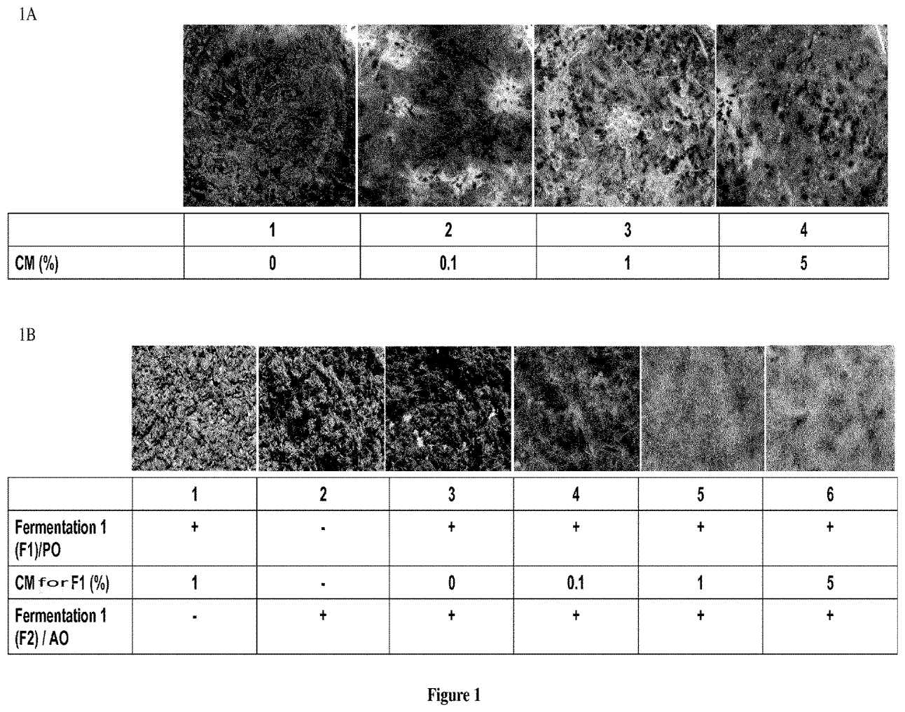

The model selected for the development of the method is based on the use of oak sawdust which is ground coarsely and subjected to a hot aqueous extraction. After adjustment of the moisture by filtration and sterilisation, the substrate is inoculated by Pleurotus ostreatus and maintained for 40 days at 28° C. to allow the development of the fungus. The growth of Pleurotus ostreatus on oak sawdust can be optimised by adding a natural and easily available alkalinising mineral supplement (ash). FIG. 1A has different culture conditions achieved in the absence or in the presence of the mineral supplement.

After 40 days of culture, Pleurotus ostreatus is easily grown on oak sawdust in the absence of mineral supplement (CM 0%) while the growth, visible by the extension of white mycelium, increases with the mineral supplement percentage until being stabilised at about 5% of CM. The use of a grinder thus makes it possible to homogenise the fermented product while the inactivation by heating makes it possible to prevent a new growth of oyster mushrooms. After these treatment conditions, the fermented substrate regains an appearance, a brown colour characteristic of wood (mycelium is no longer visible to the naked eye) (see FIG. 1A, column 3 and FIG. 1B, column 1) and the growth is ineffective without any new inoculation (FIG. 1B, column 1).

Aspergillus oryzae spores are added to the fermented sawdust then the moisture level is brought to a value between 60 and 70% before initiating a second fermentation for 3 days at 30° C. in a wet chamber. FIG. 1B shows the growth of Aspergillus oryzae from this second fermentation. This is undetectable if the spores are added on sawdust not having been subjected to a first fermentation (FIG. 1B, column 2) and its level is correlated positively to the growth level of Pleurotus ostreatus obtained during the first fermentation (comparing the quantity of white mycelium in FIG. 1A, columns 1 to 4 and FIG. 1B, columns 3 to 6).

These results therefore show that the growth of Aspergillus oryzae on only oak sawdust is dependent on a first fermentation by Pleurotus ostreatus.

A certain number of factors could explain this dependence.

On the one hand, a decrease in the lignin content is expected given the lignivorous character of Pleurotus ostreatus, a decrease and partial degradation facilitating the access of holocellulose to the enzymes secreted by Aspergillus oryzae. To this is added a partial degradation of holocellulose by Pleurotus ostreatus itself leading to the release of reducing sugars easily assimilable by Aspergillus oryzae. Measuring reducing sugars after the first fermentation has been evaluated at 20 mg/g of dry fermented product against 2 mg/g of dry sawdust before fermentation. It has been estimated at 10 mg/g of dry fermented product from the second. These results therefore show that the first fermentation allows the release of reducing sugars which could be partially used during the second fermentation for the growth of Aspergillus oryzae.

On the other hand, it is possible that certain compounds secreted by the fungus serve as an additional carbon source (such as organic acids) and nitrogen source (proteins synthesised by Pleurotus ostreatus).

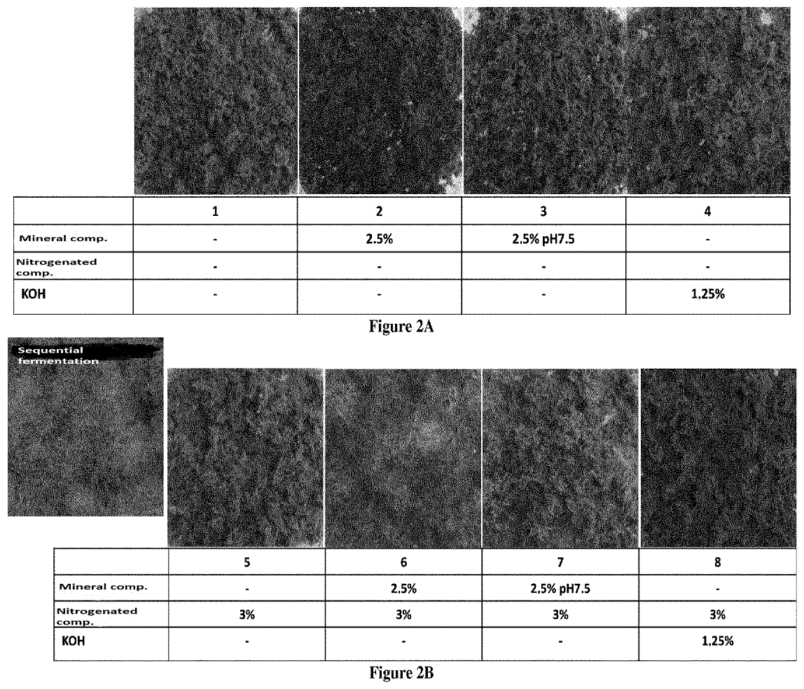

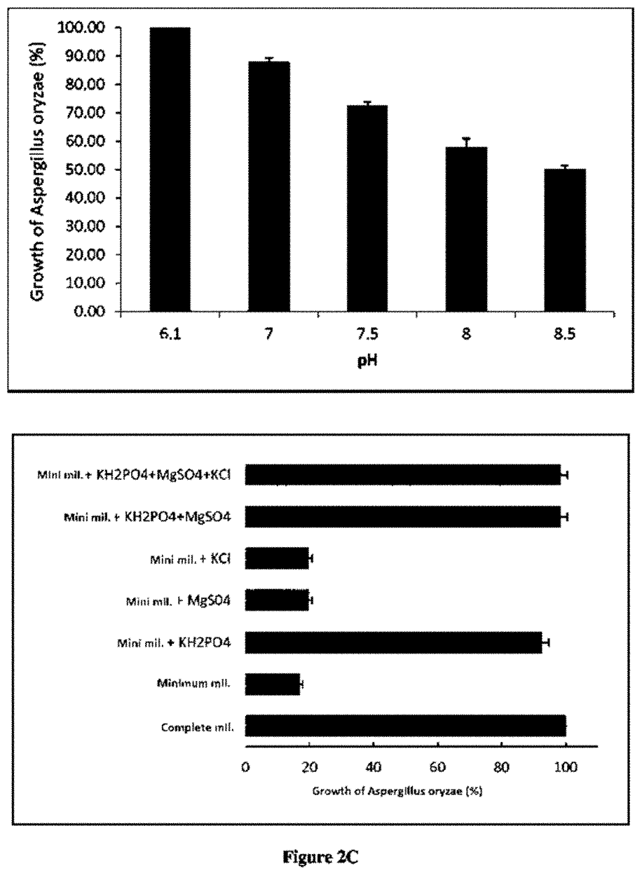

The minimum conditions for fermenting oak sawdust by Aspergillus oryzae are presented in FIG. 2 and make it possible to highlight the relevance of the sequential method on oak sawdust. Wood sawdust has been subjected to a pre-treatment similar to that performed before inoculation by Pleurotus ostreatus, namely a coarse grinding then an extraction in an aqueous medium at 90° C. After sterilisation (autoclaving), a new grinding is carried out before inoculation by the Aspergillus oryzae spores and incubation for 3 days at 30° C. As FIG. 2A shows, no growth is observable only on pre-treated wood (column 1), on pre-treated wood combined with 2.5% of alkalinising mineral supplement and 2.5% of neutral mineral supplement (here, ash, of which the pH has been adjusted to 7.5 by adding HCl) (columns 2 and 3), on pre-treated wood combined with 1.25% of alkalinising potash (column 4) and on pre-treated wood combined with 3% of nitrogenated supplement (column 5). However, growth is observed on pre-treated wood after addition of 2.5% of alkalinising or neutral mineral supplement and 3% of nitrogenated supplement (proteins) (columns 6 and 7). The growth level observed is greater when the mineral supplement is alkalinising compared with the neutral mineral supplement, but less than that observed after a first fermentation by Pleurotus ostreatus (comparing the inset and columns 6 and 7). These results suggest therefore that Aspergillus oryzae can be grown on oak sawdust in the presence of a mineral supplement and a nitrogenated supplement with a preferable development at alkaline pH. The combination of the nitrogenated supplement and alkalinising potash does not make it possible to see a development of Aspergillus oryzae on oak sawdust (column 8), confirming a dependence on the presence of mineral elements, absent in this experimental condition. The optimal growth conditions of A. oryzae combining the nitrogenated and alkalinising mineral supplements are not suitable with the published data having a growth optimum pH for this ascomycete between 6 and 7.5 (Krijgshield et al., 2013). FIG. 2B confirms a decrease of growth of A. oryzae at alkaline pH (the pH of the pre-treated wood combined with 2.5% of alkalinising mineral supplement is about 8.0) and therefore suggests that the positive effect of the alkalinisation observed on the growth Aspergillus would result in an effect on the wood (FIG. 2A, column 7) which weakens the lignocellulose of the wood and would facilitate its degradation by the enzymes secreted by Aspergillus (Rabemanolontsoa & Saka, 2015). The dependence of Aspergillus oryzae regarding mineral elements present in the mineral supplement added in the form of ash is highlighted in FIG. 2C. Compared with a complete medium and a minimal medium only containing a carbon and nitrogenated source and for which a very low growth is observed, the removal of phosphate limits any significant development of Aspergillus oryzae. The removal of magnesium and of sulphate is not limiting for the growth of the fungus but leads to a sporulation suggesting an ascomycete stress (result not presented).

These results therefore show that Aspergillus oryzae is capable of being grown on oak sawdust on the condition of adding to the substrate a mineral and nitrogenated supplement, growth is favoured if the mineral supplement is alkalinising with an effect which could be attributed to a weakening of lignocellulose (during the heating in the presence of the mineral supplement). However, the growth effectiveness is less than that observed during the sequential fermentation method.

In their entirety, these results show that the growth of Aspergillus oryzae on oak sawdust (without addition) is dependent on a first fermentation by Pleurotus ostreatus and suggest that this first fermentation, by weakening the wood and in particular, lignin, make available nutritional elements necessary for its development of which very probably, a nitrogen source which is accessible and essential for the growth of Aspergillus oryzae as well as the mineral elements essential to its development, in particular phosphate.

Effect of the Mineral Supplement on the Conduct of the Method According to the Invention

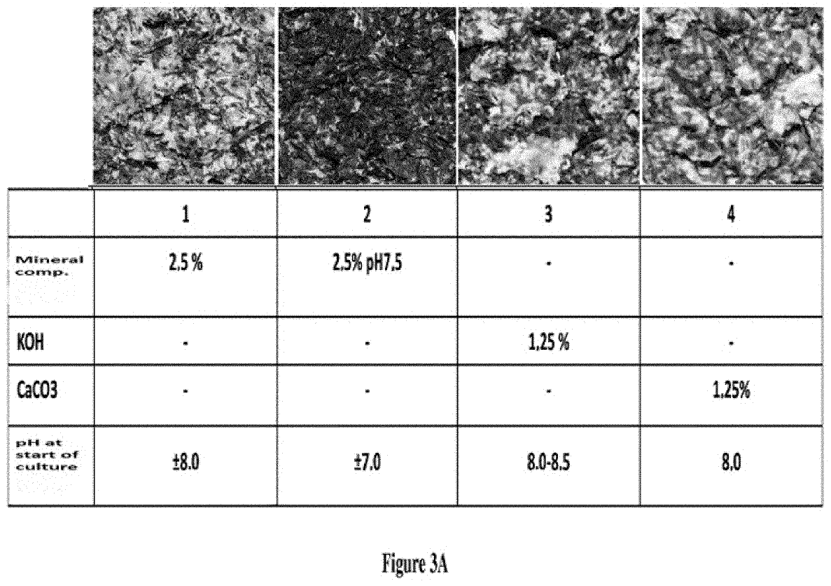

The relevance of the sequential fermentation method can also be highlighted through the analysis of the impact of the alkalinising mineral supplement on the fermentation by Pleurotus ostreatus. FIG. 3A presents the growth of Pleurotus ostreatus on oak sawdust combined with 2.5% of mineral supplement (column 1), at 2.5% of mineral supplement of which the pH has been adjusted to 7.5 (column 2), to 1.25% of potash (column 3) and to 1.25% of calcium carbonate (column 4) (1.25% of KOH have been added as ash contains about 50% of CaO mainly responsible for the alkalinity). The results obtained show that potash or calcium carbonate can be substituted for ash (comparing columns 1, 3 and 4). However, the adjustment of the mineral supplement at pH 7.5 leads to a notable decrease in the growth of Pleurotus on oak sawdust with however a greater growth of oyster mushrooms under these conditions to that observed without adding any mineral supplement (comparing with FIG. 1, column 1).

Thus, these results show that the alkalinity of the ash has a more determining effect on the growth of oyster mushrooms than the addition of minerals.

It is probable that the alkalinising effect here also works on wood and not on the growth of the fungus, itself. Indeed, different studies have shown an optimum pH for the growth of oyster mushrooms between 5 and 7 during the culture on a synthetic medium or straw (Romero-Arenas et al., 2012, Tripathi and Yadav, 1992, Belletini et al., 2016).

Finally, FIG. 3B presents the growth of Aspergillus oryzae in secondary fermentation under these experimental conditions. More surprisingly, these results show that the presence of additional mineral elements is not essential to the growth of Aspergillus oryzae during the second fermentation since the ash can be substituted by potash or calcium carbonate (comparing FIG. 3B, columns 1, 3 and 4) and reinforce the importance of the quality of the first fermentation on the second by making available nitrogenated nutritional elements and minerals taken from wood for Aspergillus oryzae.

The Method According to the Invention can be Applied to Different Wood Species

Complementary experiments have been carried out on different wood species. The species models have been selected according to their level of harvesting, their classification as species of value and their immediate availability: spruce, beech and nannyberry have thus served as a study model. Beech is the third most harvested hardwood after oak and poplar, spruce forms part of the most harvested conifers, and mountain ash is a valuable species.

The experimental conditions are based on the reference protocol developed on oak.

The pre-treatment conditions of wood, the conditions of first fermentation (with and without alkaline) and of second fermentation are similar to those used for oak.

The effectiveness of the method has been evaluated from the second fermentation by measuring xylanase, protease and amylase activities and presented in table 1. To facilitate the comparison of the results, these have been standardised to the values obtained during the use of oak as a substrate (1 arbitrary unit).

TABLE 1
Enzymatic activities measured from the sequential fermentation
method using sawdust coming from different wood species.

| | Enzymatic activity (arbitrary unit) | | | | | |
|---|---|---|---|---|---|---|
| | Amylase | | Xylanase | | Protease | |
| | 0% | 2.5% | 0% | 2.5% | 0% | 2.5% |
| | alkaline | alkaline | alkaline | alkaline | alkaline | alkaline |
| | supplement | supplement | supplement | supplement | supplement | supplement |
| Oak | 0 | 1 | 0.05 | 1 | 0 | 1 |
| Beech | 0.57 | 0.91 | 0.194 | 0.62 | 0.18 | 1.4 |
| Nannyberry | 0.54 | 0.91 | 0.25 | 0.84 | 0.21 | 1.12 |
| Spruce | 0.44 | 0.8 | 0.18 | 0.58 | 0.05 | 0.62 |

The results obtained show that i) the method developed on oak sawdust can be applied to other species with ii) a significant improvement in the production of amylase, xylanase and protease activities during the second fermentation if the method is conducted in the presence of alkaline supplement during the step of pre-treating sawdust before the first fermentation, that iii) the production of these activities is less dependent on the addition of the alkaline supplement for oak, mountain ash, and spruce species compared with oak, iv) the production of amylase, xylanase and protease is generally less when spruce is used as a substrate.

The Pre-Treatment of Water has No Damaging Effect on the Xylanase and Amylase Content of the Product Obtained by the Method According to the Invention

Complementary experiments have been carried out in order to evaluate the possible effect of different pre-treatment conditions.

The effect of the extraction temperature has been evaluated first. For this, wood (oak) residues mixed with water have been heated to 50° C., 90° C. and 120° C. or have been mixed with water without heating before proceeding with the filtration step, then adding alkaline supplement before sterilisation before inoculation by Pleurotus ostreatus for the first fermentation. The effectiveness of the method has been evaluated from the second fermentation by measuring xylanase and amylase activities. The two fermentation steps have been conducted as described above for oak residues.

No significant difference has been observed on the level of secretion of the amylase and xylanase activities from the second fermentation, confirming the possibility of using a quite wide temperature range during the extraction step.

Impact of Moisture on the Substrate on the Conduct of the Method According to the Invention

Complementary experiments have been carried out in order to confirm the impact of the moisture content of the substrate on the sequential fermentation method. For this, the moisture level of the substrate (oak residues such as used in the reference method) from the filtration has been adjusted to 40%, 50%, 60%, 70% and 80% before adding the alkaline supplement, sterilisation and inoculation. The effectiveness of the method such as described above has been evaluated from the second fermentation by measuring the xylanase and amylase activities. No significant difference has been observed on the level of secretion of the amylase and xylanase activities from the second fermentation, confirming the possibility of using the moisture levels between 40 and 80%.

Implementation of the Method According to the Invention with Other Basidiomycete Strains

The sequential fermentation method has been implemented according to the protocol described above with Pleurotus pulmonarius and Hypsizygus ulmarius substituting for Pleurotus ostreatus. As above, the effectiveness of the method has been evaluated from the second fermentation by measuring the xylanase and amylase activities secreted by Aspergillus oryzae. The table below presents the results obtained from these activities. To facilitate the comparison of the results, these have been standardised to the values obtained during the use of Pleurotus ostreatus for the first fermentation (1 arbitrary unit).

TABLE 2
Measurement of the xylanase and amylase activities from
the fermentation by Aspergillus oryzae according
to the basidiomycete used for the first fermentation.

Enzymatic activity (arbitrary unit)

| | Amylase | Xylanase |
|---|---|---|
| Pleurotus ostreatus | 1 | 1 |
| Pleurotus pulmonarius | 1 | 1.25 |
| Hypsizygus ulmarius | 0.92 | 0.56 |

The results obtained show that the sequential fermentation method according to the invention can be applied to Pleurotus pulmonarius and Hypsizygus ulmarius, two wood destroying fungi not leading to a significant difference on the production of amylase by Aspergillus oryzae.

The regulation of the secretion of the xylanase activities has been widely studied and this is controlled by the respective levels of inducers (xylan, xylose with low concentration, nitrogen, etc.) and repressors (xylose with high concentration, glucose, etc.) potentially present in the medium. It is possible that the first fermentation conducted at the production/release of inducing compounds and/or repressors varying according to the fungus species used, which would explain the difference of production of xylanase when the first fermentation is carried out in the presence of Hypsizygus ulmarius.

In conclusion, the sequential fermentation method can be applied to different wood destroying fungi species by ensuring the level of secretion of the enzymatic activities sought.

Implementation of the Method According to the Invention with Aspergillus awamori and in Coculture of Aspergillus oryzae and Aspergillus awamori

The sequential fermentation method has been applied to Aspergillus awamori, a mould of the Aspergillus genus used in traditional Japanese food.

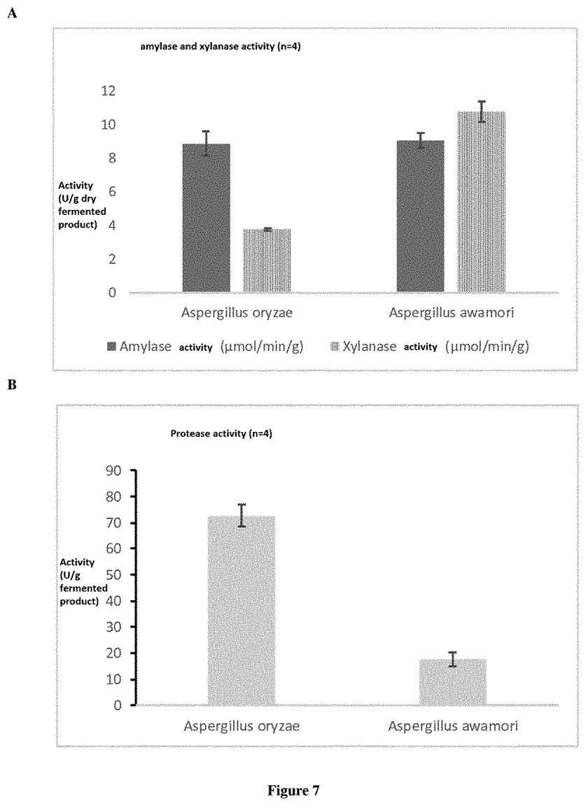

The steps of pre-treating oak sawdust then of inoculation by Pleurotus ostreatus have been carried out according to the reference protocol. The substrate from the first fermentation has been adjusted to 65% moisture level before being inoculated by 2·10⁶ Aspergillus awamori or Aspergillus oryzae spores per gram of dry fermented product then incubated for 3 days at 30° C. (reference protocol). The effectiveness of the method has been evaluated by measuring xylanase, amylase and protease activities from the second fermentation. The results obtained are presented in FIG. 7. They are expressed in U/g of dry fermented product.

These show that the secretion of amylase by Aspergillus oryzae and awamori is comparable (panel A). However, the xylanase and protease activities are significantly different between the two species. The xylanase activity is greater for Aspergillus awamori (about 2.5 times) (panel A) while the protease activity is less for A. awamori (about 3.5 times) (panel B).

Taken together, these results show i) that another species of the Aspergillus genus can be grown on fermented wood residues by following the steps established for Aspergillus oryzae, making it possible to propose that the method is applicable to other species of the Aspergillus genus, ii) that the enzymatic activities sought initially, namely the xylanase, amylase and protease activities are present from the fermentation for the two species used, awamori and oryzae and iii) that the level of secretion of the xylanase and protease activities is variable according to the species considered.

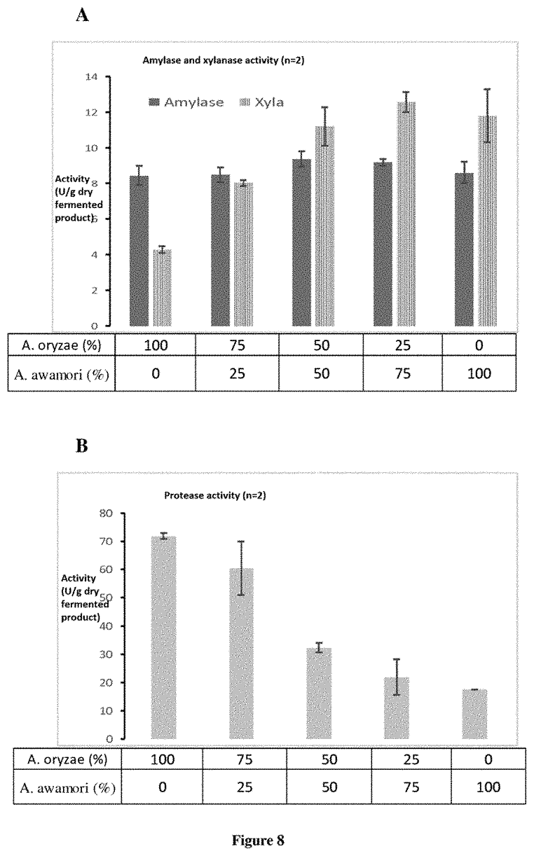

According to this last observation, modulating the level of the xylanase and protease activities by combining the species can be contemplated. FIG. 8 shows the results of the measurements of the amylase, xylanase and protease activities obtained by producing Aspergillus awamori and Aspergillus oryzae cocultures during the second fermentation, the percentage of each mould varying between 100, 75, 50 and 25% of the coculture. As expected, the level of secretion of the amylase activities is similar whatever the respective percentage of A. oryzae or awamori (panel A). The level of secretion of xylanase activity increases when the percentage of Aspergillus awamori increases to be stabilised when the A. oryzae/A. awamori ratio is identical (about 12 U/g of fermented product) (panel A).

Correlating to the results presented in FIG. 7, the level of protease activity decreases when the percentage of A. awamori increases, this decrease being significant from an identical A. awamori/A. oryzae ratio (50/50). In their entirety, these results show that i) the coculture of moulds of the Aspergillus genus on the fermented wood residues can be considered and ii) a controlled coculture at the moment of the inoculation makes it possible to modulate the respective level of secreted enzymes. For example, A. oryzae can be used by itself, if the protease activities are sought and combined with A. awamori in order to increase the level of secretion of the xylanase activities.

The First Fermentation of the Method Leads to the Production of Laccase by P. ostreatus

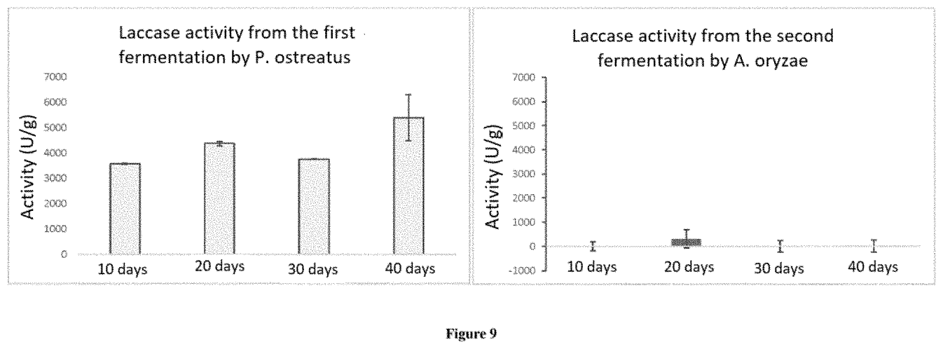

The presence of laccase activity is detected from the first fermentation; however, this enzyme is hardly or not detected from the second fermentation (see FIG. 9).

Knowing that this activity is responsible for the degradation of lignin and that the lignin content of the wood from the method is 12% (against a theoretical content between 17 and 25% before fermentation), the decrease of the lignin content can be associated with this first fermentation.

The Sequential Fermentation Allows the Production of Xylanases, Amylases and Proteases by Aspergillus oryzae

The enzymes secreted by filamentous fungi are widely used in animal food to improve the digestibility of food and to increase the growth performances of farm animals (Asmare, 2014). Aspergillus oryzae has been used in human food for several millennia and described for its capacity to synthesise and to secrete enzymes involved in the degradation of starch-rich and also lignocellulosic substrates (Brink & Vries, 2011; Kobayashi et al., 2007; Vries & Visser, 2001).

The xylanase, protease, amylase, glucanase and phytase activities are the activities the most sought for animal food (Shallom & Shoham, 2003; Kuhad et al., 2011; Asmare, 2014). Assays for xylanase, protease and amylase activities have been developed in order to evaluate the level of secretion of these enzymes by Aspergillus oryzae while comparing it to that of Pleurotus ostreatus.

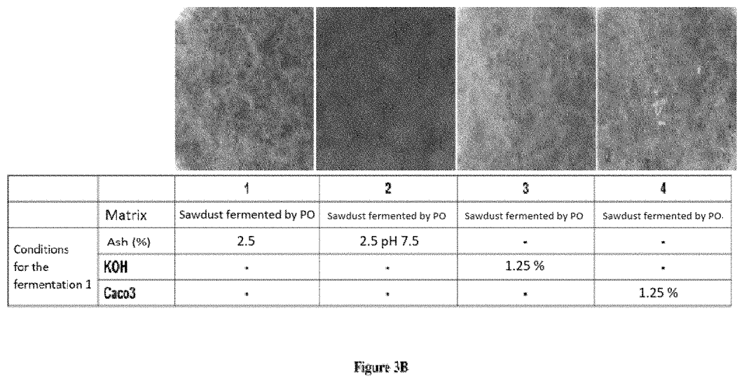

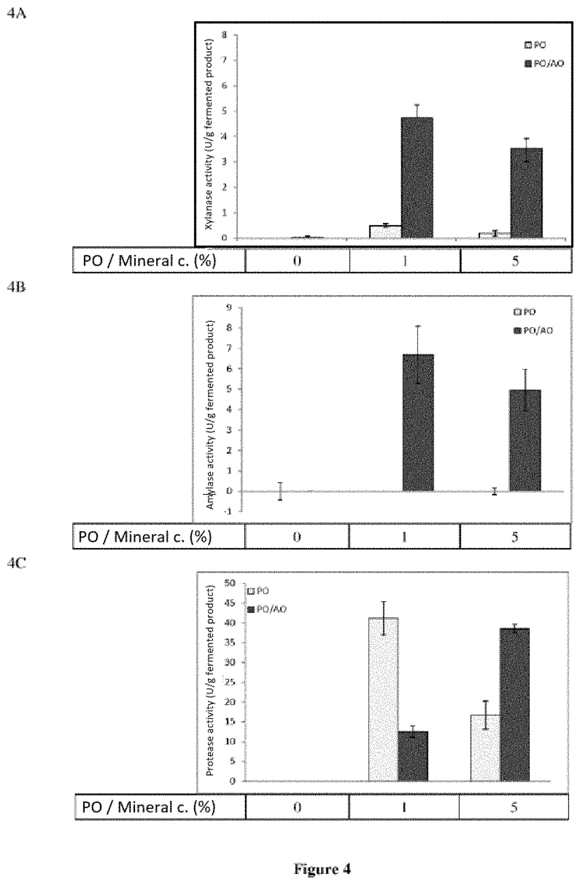

FIG. 4 presents the xylanase, amylase and protease activities secreted by Pleurotus ostreatus from the first fermentation and Aspergillus oryzae from the second. Three culture conditions have been compared, one carried out without adding any mineral supplement and two carried out with the addition of 1 and 5% of mineral supplement, two conditions stimulating the development of Pleurotus ostreatus and consequently that of Aspergillus oryzae.

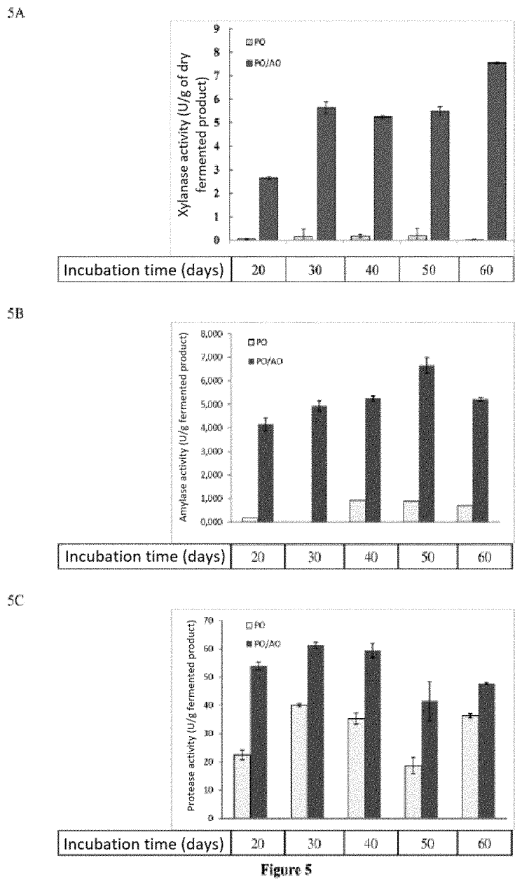

Thus, if the fermentation carried out by Pleurotus ostreatus is a condition sine qua non to the growth of Aspergillus oryzae, the second fermentation gives the fermented product, added value in particular through the presence of digestive enzymes. It is important to note that the level of secretion of these enzymes by Pleurotus ostreatus remains much less than that of Aspergillus oryzae whatever the duration of the first fermentation. FIGS. 5A and B shows almost zero secretion of xylanases and amylases after 20, 30, 40, 50 and 60 days of fermentation by Pleurotus ostreatus in the presence of 2.5% of ash. The xylanase and amylase activities secreted by Aspergillus oryzae increase between 20 and 30 days of first fermentation (by Pleurotus ostreatus) to be stabilised then (the incubation duration of Aspergillus oryzae remained constant for 3 days). The differences in the levels of secretion of the protease activities are less pronounced between Pleurotus ostreatus and Aspergillus oryzae whatever the duration of the first fermentation, but are always in favour of Aspergillus oryzae under the conditions where the mineral supplement has been added at a level of 2.5% (see FIG. 5C) and 5% (see FIG. 4C).

Taken together, these results show that the sequential fermentation method on wood residues using Pleurotus ostreatus then Aspergillus oryzae allows the production of enzymes, such as xylanases, amylases and proteases.

Sequential Fermentation Allows the Degradation of Lignin.

One of the obstacles to using lignocellulosic compounds and particularly wood for animal food is its rigidity due to its lignin content of about 25% (Guerriero et al., 2016).

The lignin content of the fermented product from the sequential fermentation has been evaluated and represents 11% of lignin, value a lot less than the lignin content of the wood residues (starting product).

The Enzymatic Activities of the Fermented Product can be Stabilised on the Substrate.

The secretion of digestive enzymes during the second fermentation opening up prospects of enhancing fermented wood sawdust, it appears essential to develop a method for stabilising and for conserving these simple and inexpensive enzymes.

In the method implemented, from the second fermentation, the fermented product of which the moisture level is close to 60% is made homogenous by mechanical stirring then placed in a chamber at 24° C. until obtaining a moisture content of 12%, the moisture level stabilising the product from a microbiological standpoint and preventing an increase in growth or the development of other types of microorganisms (Assamoi et al., 2009).

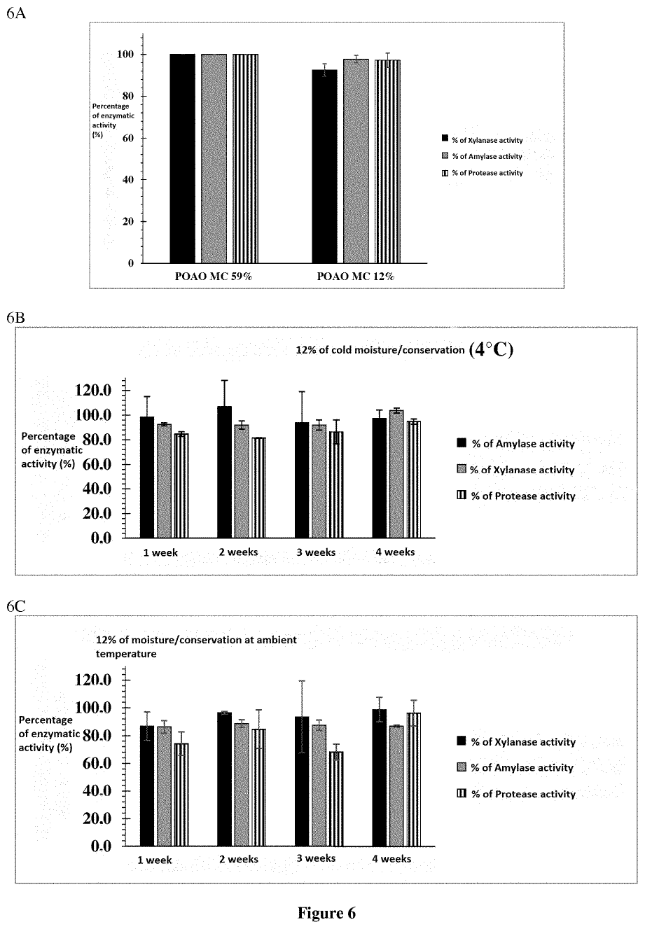

FIG. 6A presents the residual enzymatic activities after dehydration to 12% and shows that dehydration carried out under these conditions does not lead to any loss of xylanase, amylase and protease activities.

FIG. 6B presents the same residual activities after conservation at 4° C. of the fermented and dehydrated product for one, two, three or four weeks. No significant loss of activity is observable whatever the activities measured.

FIG. 6C presents the residual xylanase, amylase and protease activities after conservation at ambient temperature of the fermented and dehydrated product for one, two, three and four weeks. As above, no significant loss of activity is observable whatever the activities measured.

These results therefore confirm the possibility of using the fermented substrate as a support for stabilisation/immobilisation of the secreted enzymes.

The beta-glucan content (main compounds of the wall of the filamentous fungi) present in the final fermented product is measured at about 8%.

### 3. CONCLUSION

The experimental data highlight the production of a fermented product of food quality from wood; this product is complex and composed of residual digested lignocellulose (in a proportion similar to that of straw), mycelium and compounds secreted by fungi comprising enzymes. These enzymes of interest are usually added to the animal food ration (Asmare, 2014). In current methods, the purified enzymes must be “diluted” by mixing with mineral meals or matrices before being incorporated in the food. The enzymes secreted and stabilised on the final fermented product according to the invention are already “diluted” by the presence of the residual substrate and of mycelium, a “premixing” with a meal or a matrix can be avoided, facilitating the production and limiting the production cost of the enriched food.

### BIBLIOGRAPHIC REFERENCES

Agreste. (2012). Forests and the Forests sector sector Key data on: «Forestedl areas 2012 Edition, (May).
Alexandre, S. (2017). Rapport de mission de la dellelguele interministelrielle a la fore t et au bois.
Asmare, B. (2014). Effect of common feed enzymes on nutrient utilization of monogastric animals. 5(July), 27-34. https://doi.org/10.5897/IBMBR2014.0191
Assamoi, A. A., Destain, J., & Thonart, P. (2009). Aspects microbiologiques de la production par fermentation solide des endo-β-1, 4-xylanases de moisissures: le cas de Penicillium canescens, 13(2), 281-294.
Bowman, S. M., & Free, S. J. (2006). The structure and synthesis of the fungal cell wall. BioEssays: News and Reviews in Molecular, Cellular and Developmental Biology, 28(8), 799-808. https://doi.org/l0.1002/bies.20441
Brink, J. Van Den, & Vries, R. P. De. (2011). Fungal enzyme sets for plant polysaccharide degradation. Applied Microbiology. 1477-1492. https://doi.org/10.1007/s00253-11-3473-2
Chancharoonpong, C., Hsieh, P.-C., & Sheu, S.-C. (2012). Production of Enzyme and Growth of Aspergillus oryzae S. on Soybean Koji. International Journal of Bioscience, Biochemistry and Bioinformatics, 2(4), 228-231. https://doi.org/10.7763/IJBBB.2012.V2.106
Dashtban, M., Schraft, H., & Qin, W. (2009). Fungal Bioconversion of Lignocellulosic Residues: Opportunities & Per-spectives. Review Literature And Arts Of The Americas, 5(6), 578-595.
FAO. (2016). Situation des Forêts.
Ferreira, J. A., Mahboubi. A., Lennartsson, P. R., & Taherzadeh, M. J. (2016). Bioresource Technology Waste biorefineries using filamentous ascomycetes fungi: Present status and future prospects. Bioresource Technology, 215, 334-345. https://doi.org/10.1016/j.biortech.2016.03.018
Gilani, G. S., Xiao, C. W., & Cockell. K. A. (2017). Impact of Antinutritional Factors in Food Proteins on the Digestibility of Protein and the Bioavailability of Amino Acids and on Protein Quality, (2012). https://doi.org/10.1017/S007114512002371
Girmay, Z., Gorems, W., Birhanu, G., & Zewdie, S. (2016). Growth and yield performance of Pleurotus ostreatus (Jacq. Fr.) Kumm (oyster mushroom) on different substrates. AMB Express, 6(1), 87. https://doi.org/10.1186/s13568-016-0265-1
Guerriero, G., Hausman, J.-F., Strauss, J., Ertan, H., & Siddiqui. K. S. (2016). Lignocellulosic biomass: Biosynthesis, degradation, and industrial utilization. Engineering in Life Sciences, 16(1), 1-16. httpsJ/doi.org/10.1002/elsc.201400196
Hoa, H. T., & Wang, C. (n.d.). Mycobiology The Effects of Temperature and Nutritional Conditions on Mycelium Growth of Two Oyster Mushrooms (Pleurotus ostreatus and Pleurotus cystidiosus), 14-23.
Hoa, H. T., Wang. C., & Wang, C. (n.d.). Mycobiology The Effects of Different Substrates on the Growth, Yield, and Nutritional Composition of Two Oyster Mushrooms Pleurotus ostreatus and Pleurotus cystidiosus). 423-434.
Howard, R. L., Abotsi, E., L, J. V. R. E, & Howard, S. (2003). Lignocellulose biotechnology: issues of bioconversion and enzyme production, 2(December), 602-619.
Janser, R., Castro, S. De. & Sato, H. H. (2014). Protease from Aspergillus oryzae: Biochemical Characterization and Application as a Potential Biocatalyst for Production of Protein Hydrolysates with Antioxidant Activities, 2014.
Kobayashi, T., Abe, K., Asai, K., Gomi, K., Juvvadi, P. R., Kato, M., . . . Machida, M. (2007). Genomics of Aspergillus oryzae. Bioscience, Biotechnology, and Biochemistry, 71(3). 646-70. https://doi.org/10.1271/bbb.60550
Krajewska, B. (2014). Enzyme immobilization by adsorption: a review MODIFIER ENZYME, 801-821. https://doi.org/10.1007/s10450-014-9623-y
Krijgsheld, P., Bleichrodt, R., van Veluw, G. J., Wang, F., Miiller, W. H., Dijksterhuis, J., & Wösten, H. a B. (2013). Development in Aspergillus. Studies in Mycology, 74(1), 1-29. https://di.org/10.3114/sim( )06
Kuhad, R. C., Gupta. R., & Singh, A. (2011). Microbial Cellulases and Their Industrial Applications. Enzyme, 2011. https://doi.org/10.4061/2011/280696
Plácido, J., & Capareda, S. (2015). Ligninolytic enzymes: a biotechnological alternative for bioethanol production. ??? https:/doi.org/10.1186/s40643-015-0049-5
Rabemanolontsoa, H., & Saka, S. (2015). Various preteatments of lignocellulosics. Bioresource Technology, 199, 83-91. https:/doi.org/10.1016/j.biortech.2015.08.029
Ravindran, R., & Jaiswal, A. K. (2015). A comprehensive review on pe-treatment strategy for lignocellulosic food industry waste: Challenges and opportunities. Bioresource Technology, 199, 92-102. https:/doi.org/10.1016/j.biortech.2015.07.106
Saritha, M., Arora, A., & Lata. (2012). Biological pretreatment of lignocellulosic substrates for enhanced delignification and enzymatic digestibility. Indian Journal of Microbiology, 52(2), 122-30. https://doi.org/10.1007/s12088-011-0199-x
Shallom, D., & Shoham, Y. (2003). Microbial hemicellulases. Current Opinion in Microbiology, 6(3), 219-228. https://doi.org/10.1016/S1369-5274(03)00056-0
Sharma, R. K., & Arora, D. S. (2015). Fungal degradation of lignocellulosic residues: an aspect of improved nutritive quality. Critical Reviews in Microbiology, 41(1), 52-60. https://doi.org/10.3109/1040841X.2013.791247
Soccol. C. R., Scopel, E., Alberto, L., Letti, J., Karp, S. G., Woiciechowski, A. L., . . . Vandenberghe, D. S. (2017). Recent developments and innovations in solid state fermentation. Biotechnology Research and Innovation. https://doi.org/10.1016/j.biori.2017.01.002
Song. M. H., Nah, J., Han, Y. S., Han, D. M., & Chae, K. (2001). Promotion of conidial head formation in Aspergillus oryzae by a salt, 80, 689-691.
Szilagyi, M Kwon, N., Dorogi, C., Pocsi, I., Yu, J.-H., & Emri T. (2010). The extracellular b-1, 3-endoglucanase EngA is involved in autolysis of Aspergillus nidulans. 1498-1508. https://doi.org/10.1111/j.1365-2672.2010.04782
Thomas, L., Larroche, C., & Pandey, A. (2013). Current developments in solid-state fermentation. Biochemical Engineering Journal, 81.146-161. https://doi.org/10.1016/j.bej.2013.10.013
Valásková, V., & Baldrian, P. (2006). Degradation of cellulose and hemicelluloses by the brown rot fungus Piptoporus betulinus—production of extracellular enzymes and characterization of the major cellulases. Microbiology (Reading, England), 152(Pt 12), 3613-22. https://doi.org/10.1099/mic.0.29149-0
van den Brink, J., van Muiswinkel, G. C. J., Theelen, B., Hinz, S. W. a, & de Vries, R. P. (2013). Efficient plant biomass degradation by thermophilic fungus Myceliophthora heterothallica. Applied and Environmental Microbiology, 79(4), 1316-24. https://doi.org/10.1128/AEM.02865-12
Volman, J. J. Ramakers, J. D., & Plat, J. (2008). Dietary modulation of immune function by β-glucans. 94.276-284. https://doi.org/10.1016/j.physbeh.2007.11.045
Vries, R. P. De, & Visser, J. (2)1). Aspergillus Enzymes Involved in Degradation of Plant Cell Wall Polysaccharides Aspergillus Enzymes Involved in Degradation of Plant Cell Wall Polysaccharides. 65(4). https://doi.org/10.1128/MMBR.65.4.497
Wertz. J. L. (2010). Note de synthèse (22 Nov. 2010).

1. A method for transforming wood residues into edible food product for a mammal, the method comprising the steps of:
1) performing a first fermentation of a substrate composed of wood residues by a wood destroying edible fungus for a suitable duration corresponding to the maximum colonisation of the substrate before fructification by said fungus;
2) stopping the first fermentation by heat inactivation of said wood destroying edible fungus and grinding a product obtained from said first fermentation;
3) performing a second fermentation of the product obtained in step 2) by a fungus of the Aspergillus genus for a suitable duration corresponding to the maximum colonisation of the substrate before sporulation by said fungus of the Aspergillus genus.

2. The method for transforming wood residue into an edible food product for a mammal according to claim 11, wherein the pre-treatment step before step 1) comprises grinding of wood residues to obtain a size of wood residues less than or equal to 2 cm and/or heating at a temperature of at least 70° C. in a wet medium.

3. The method for transforming wood residues into an edible food product for a mammal according to claim 1, wherein said wood residues comprises a mixture of between 40 and 80% by weight of wood sawdust and between 20 and 60% by weight of wood meal.

4. The method for transforming wood residues into an edible food product for a mammal according to claim 12, wherein said alkalinizing mineral supplement represents an input of:
170 to 330 kg/t of calcium (expressed in the form of CaO),
20 to 60 kg/t of potassium (expressed in the form of K₂O),
25 to 46 kg/t of magnesium (expressed in the form of MgO),
10 to 61 kg/t of phosphorus (expressed in the form of P₂O₅),
metals, which are cofactors of digestive enzymes secreted by fungi,
and has a pH, before mixing with wood residues, between 10 and 13.

5. The method for transforming wood residues into edible food product for a mammal according to claim 1, wherein the wood destroying edible fungus is selected from Pleurotus ostreatus Pleurotus pulmonarius, Hypsizygus ulmarius, Agaricus blasei and Agaricus braziliensis.

6. The method for transforming wood residues into edible food product for a mammal according to claim 1, wherein said fungus of the Aspergillus genus is selected from Aspergillus oryzae, Aspergillus niger, Aspergillus sojae, and Aspergillus awamori.

7. A fermented food product obtainable by the method according to claim 1.

8. The fermented food product according to claim 7, wherein the fermented food product comprises the following composition:
between 5 and 10 U of xylanases/g of dry food product;
between 5 and 10 U of amylases/g of dry food product;
between 30 and 100 U of proteases/g of dry food product;
between 20 and 40 mg of vitamin B3/g of dry food product;
an amino acid profile comprising between 10 and 15 mg of histidine, between 30 and 45 mg of isoleucine, between 40 and 65 mg of leucine, between 20 and 30 mg of lysine, between 10 and 15 mg of methionine, between 25 and 40 mg of phenylalanine, between 12 and 19 mg of tyrosine, between 35 and 54 mg of threonine, between 25 and 40 mg of valine and between 8 and 12.5 mg of tryptophan/g of total proteins of said food product;
a lignin content less than 18%.

9. A method of supplementing the diet of an animal, comprising incorporating the food product according to claim 7 in an animal food ration provided to the animal.

10. The method of claim 9, wherein the animal is a monogastric farm animal.

11. The method for transforming wood residue into an edible food product for a mammal according to claim 1, further comprising pre-treating the wood residues prior to the first fermentation of step 1).

12. The method for transforming wood residue into an edible food product for a mammal according to claim 1, wherein the substrate composed of wood residues in the first fermentation of step 1) further comprises 1 to 5% by dry weight of an alkalinizing mineral supplement.

13. The method for transforming wood residue into an edible food product for a mammal according to claim 1, further comprising stabilizing a product obtained from said second fermentation of step 3) by dehydration.

14. The method for transforming wood residue into an edible food product for a mammal according to claim 4, wherein the metals comprise one or more of Mn, Fe, Cu, and Zn in any proportion.
